(19) United States
(12) Patent Application Publication
(10) Pub. No.: US 2006/0241552 A1
(43) Pub. Date: Oct. 26, 2006

(54) Level detector of intravenous drip

(76) Inventor: Ming-Piao Tang, Taipei City (TW)

Correspondence Address: BIRCH STEWART KOLASCH & BIRCH, PO BOX 747, FALLS CHURCH, VA, 22040-0747, US

(21) Appl. No.: 11/109,690

(22) Filed: Apr. 20, 2005

(51) Int. Cl. A61M 5/00

(52) U.S. Cl. 604/253

(57) ABSTRACT

A level detector of intravenous drip comprising a shell body with a electromagnetic sensing circuitry, an alarm lamp and a buzzer installed internally, two electromagnetic inducting slices stretch out from the electromagnetic sensing circuitry to measure the liquid level inside the intravenous drip and remind the medical personnel; the shell body is fixed externally to the intravenous drip, two electromagnetic inducting slices sense the voltage difference generated by the liquid flow frequency inside the intravenous drip to measure the liquid level, the sensed voltage difference signals drive the alarm lamp and the buzzer inside the shell body for sound and light warning effect, to remind the medical personnel for following tasks, such can reduce the waste of medical resources and very convenient to install and apply.

### BACKGROUND OF THE INVENTION

I. Field of the Invention

This invention relates generally to a level detector and, more specifically, to a level detector of intravenous drip that measures the internal liquid level, especially an apparatus can be applied inside the intravenous drip.

II. Description of the Prior Art

Heretofore, it is known that the intravenous drips of medical treatment comprise a container in can shape with a needle attached, the liquid medicines are placed inside the container that is hung reversely, the liquid medicines drip from bottom of the container into patients' body through the needle by gravity; the flow quantity control is done with a simple adjustment mechanism, and by experience of those medical personnel to judge when the medicines should be replaced or removed, such might cost lot of medical resources to check the condition of the intravenous drip repeatedly; in order to achieve warning effect, some alarm systems telling medical personnel that the liquid medicine is running out are available in the market; the sensing mechanisms are done by weight sensor, floater or height measurement, currently the level height measurement is the most convenient way, referring to FIG. 1, the most popular infrared level height measurement method, a clip a1 fixes the sensor a on the external wall of the intravenous drip, an infrared transmitter a11 is inside the clip al, an infrared receiver a12 is on the other side of the clip al; when the liquid level is lower than the warning level set by the infrared transmitter all, the infrared receiver a12 issues warning signals to warning indicator a2 or buzzer a3 to remind medical personnel for following processes, the installation and application is very convenient; however the liquid medicines have refraction effect, when patients move that might cause misjudgments of intravenous drip a, and when the liquid medicines are transparent, this kind of apparatus cannot be applied.

The inventor based on above defects applies electromagnetic inducting slices to sense the voltage difference generated by the liquid flow inside the intravenous drip a to measure the liquid level.

### SUMMARY OF THE INVENTION

It is therefore a primary object of the invention to provide a level detector of intravenous drip that reduces the waste of medical resources and is very convenient to apply.

It is still an object for the invention to provide a level detector of intravenous drip in which can be applied on the industry, laboratory or other related fields to measure the liquid level inside the containers.

In order to achieve the objective set forth, a level detector of intravenous drip in accordance with the present invention comprises a shell body with a electromagnetic sensing circuitry, an alarm lamp and a buzzer installed internally, the shell body is in wristwatch shape with two fixing belts on both sides, two electromagnetic inducting slices stretch out from the electromagnetic sensing circuitry, a signal amplifier is on the electromagnetic sensing circuitry, two electromagnetic inducting slices are in different length, and are in vertical position while application, the electromagnetic inducting slices are installed internally or externally of the intravenous drip on the farthest locations along the diameter.

While application based on the structure described above, the shell body is fixed externally to the intravenous drip, the electromagnetic inducting slices sense the voltage difference generated by the liquid flow frequency inside the intravenous drip to measure the liquid level, the electromagnetic inducting slices can be fixed inside or outside at the proper location of the intravenous drip; after receiving the signals from the electromagnetic inducting slices, the signal amplifier of the electromagnetic sensing circuitry amplifies the signals to drive the alarm lamp and the buzzer inside the shell body Ad for sound and light warning effect, to remind the medical personnel for following tasks, such can reduce the waste of medical resources and very convenient to install and apply; when the present invention is applied on the industry, laboratory or other related fields, since the containers might contain erosive liquids, the known prior art with contact method will be damaged by those erosive liquids, the containers might not be transparent, the infrared detection is not applicable; the present invention applies the same method described above, the electromagnetic inducting slices of the sensor are installed on the outer wall of these containers to avoid the damage of the electromagnetic inducting slices and accurately measure the liquid level inside the containers to remind the related staff for general purpose application.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows:

FIG. 1 is a cross-sectional view of the prior art;

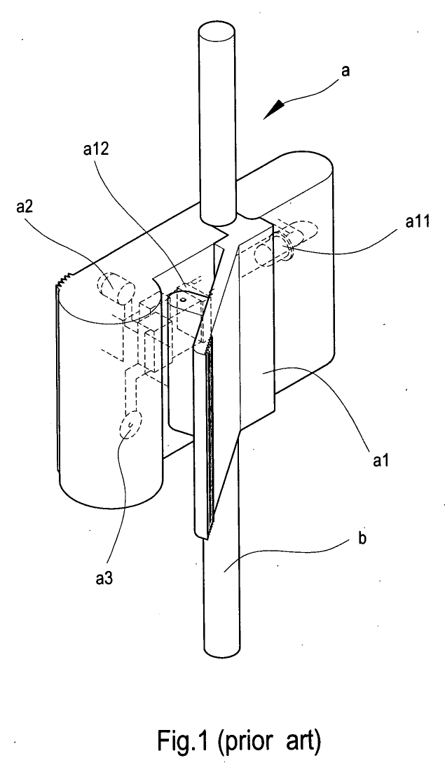

FIG. 2 is an assembly view of the present invention;

FIG. 3 is a perspective view of the present invention;

FIG. 4A is an application view of the present invention;

FIG. 4B is another application view of the present invention;

FIG. 5 is a perspective view of another application of the present invention.

### DESCRIPTION OF THE PREFERRED EMBODIMENT

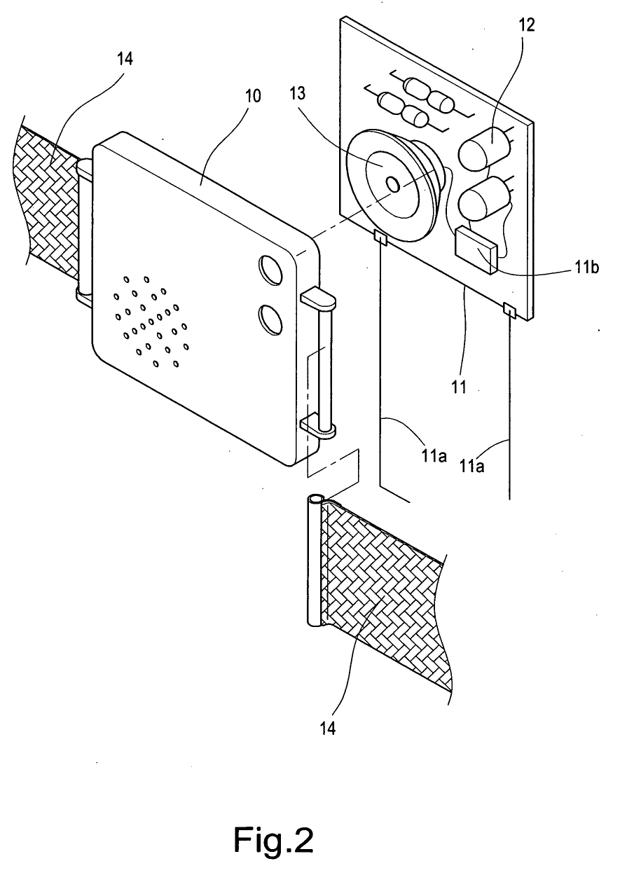

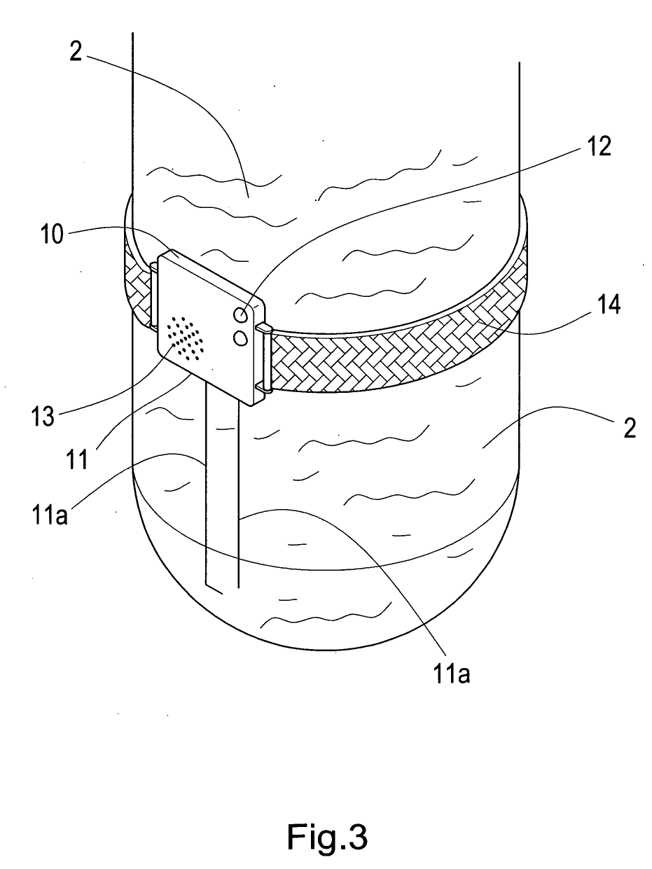

Referring to FIG. 2 and FIG. 3, the present invention comprises a shell body 10 with a electromagnetic sensing circuitry 11, an alarm lamp 12 and a buzzer 13 installed internally, the shell body 10 is in wristwatch shape with two fixing belts 14 on both sides, two electromagnetic inducting slices 11a stretch out from the electromagnetic sensing circuitry 11, a signal amplifier 11b is on the electromagnetic sensing circuitry 11, two electromagnetic inducting slices 11a are in different length, and are in vertical position while application, the electromagnetic inducting slices 11a are installed internally or on one side of the intravenous drip 2 externally, or internally or the farthest locations along the diameter.

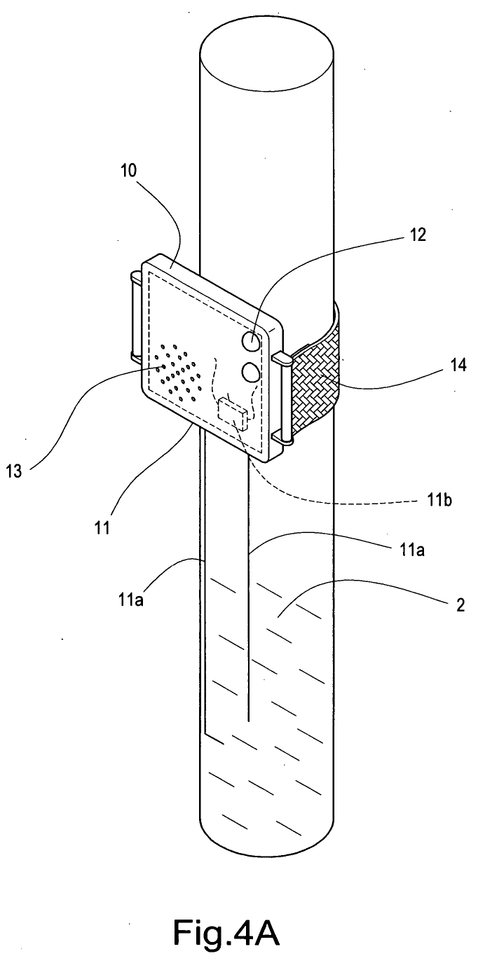

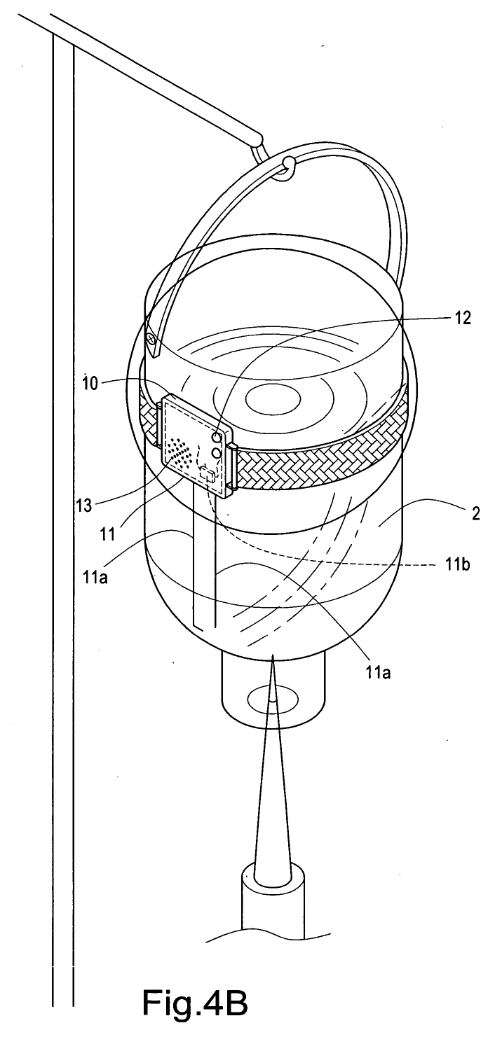

Referring to FIG. 4A and FIG. 4B, the application diagrams of the present invention, while application based on the structure described above, the shell body 10 is fixed externally to the intravenous drip 2, the electromagnetic inducting slices 11a sense the voltage difference generated by the liquid flow frequency inside the intravenous drip 2 to measure the liquid level, the electromagnetic inducting slices 11a can be fixed inside or outside at the proper location of the intravenous drip 2; after receiving the signals from the electromagnetic inducting slices 11a, the signal amplifier 11b of the electromagnetic sensing circuitry 11 amplifies the signals to drive the alarm lamp 12 and the buzzer 13 inside the shell body 10 for sound and light warning effect.

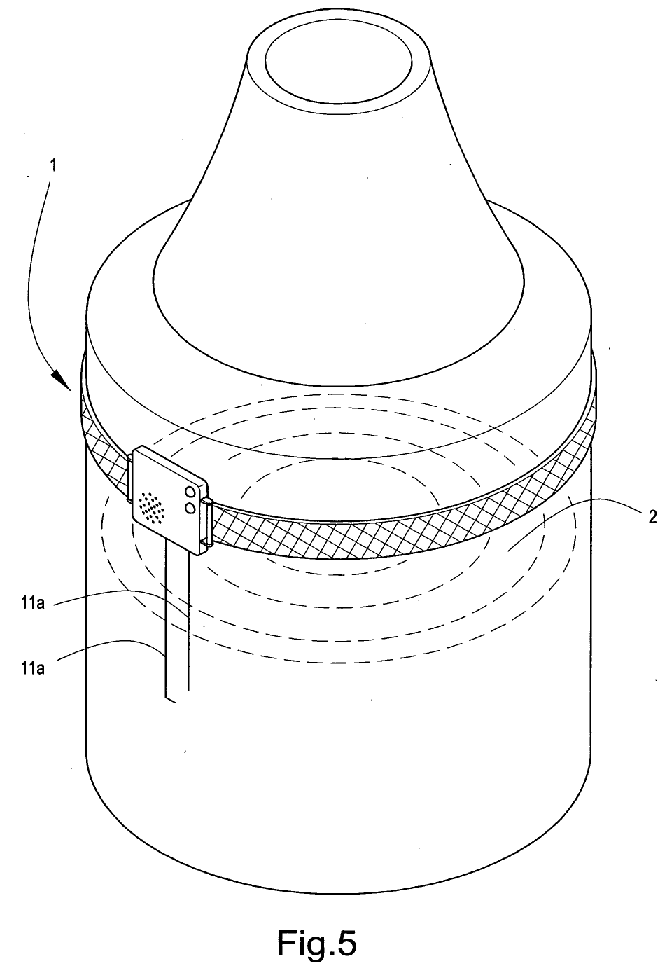

Referring to FIG. 5, another application of the present invention, when the sensor 1 of the present invention is applied on the industry, laboratory or other related fields, since the container 2 might contain erosive liquid, the electromagnetic inducting slices 11a of the sensor 1 can be installed on the outer wall of the container 2, such can avoid the damage of the electromagnetic inducting slices 11a and accurately measure the liquid level inside the container 2 to remind the related staff for general purpose application.

While a preferred embodiment of the invention has been shown and described in detail, it will be readily understood and appreciated that numerous omissions, changes and additions may be made without departing from the spirit and scope of the invention.

What is claimed is:

1. A level detector of intravenous drip comprising:
a shell body with a electromagnetic sensing circuitry, an alarm lamp and a buzzer installed internally, two electromagnetic inducting slices stretch out from said electromagnetic sensing circuitry, said shell body is fixed externally to the intravenous drip, two electromagnetic inducting slices are fixed onto the proper location of the intravenous drip to sense the voltage difference generated by the liquid flow frequency inside the intravenous drip and measure the liquid level, the sensed voltage difference signals drive said alarm lamp and said buzzer inside said shell body for sound and light warning effect.

2. The level detector of intravenous drip recited in claim 1, wherein said shell body is in wristwatch shape with two fixing belts on both sides.

3. The level detector of intravenous drip recited in claim 1, wherein a signal amplifier is on said electromagnetic sensing circuitry.

4. The level detector of intravenous drip recited in claim 1, wherein said two electromagnetic inducting slices are in different length.

5. The level detector of intravenous drip recited in claim 1, wherein said two electromagnetic inducting slices are in vertical position while application.

6. The level detector of intravenous drip recited in claim 1, wherein said electromagnetic inducting slices are installed inside the intravenous drip on the farthest locations along the diameter.

7. The level detector of intravenous drip recited in claim 1, wherein said electromagnetic inducting slices are installed outside the intravenous drip on the farthest locations along the diameter.
